(19) United States
(12) Patent Application Publication
(10) Pub. No.: US 2026/0171243 A1
(43) Pub. Date: Jun. 18, 2026

(54) SYSTEMS AND METHODS FOR RECONSTRUCTING A 3D MEDICAL REPRESENTATION BASED ON SCREEN CAPTURES

(71) Applicant: Shanghai United Imaging Intelligence Co., Ltd., Shanghai (CN)

(72) Inventors: Shanhui Sun, Lexington, MA (US); Xiao Chen, Lexington, MA (US); Terrence Chen, Lexington, MA (US)

(73) Assignee: Shanghai United Imaging Intelligence Co., Ltd., Shanghai (CN)

(21) Appl. No.: 18/986,331

(22) Filed: Dec. 18, 2024

(51) Int. Cl. G16H 50/20; G16H 15/00; G16H 30/40

(52) U.S. Cl. CPC G16H 50/20; G16H 15/00; G16H 30/40

(57) ABSTRACT

Disclosed herein are systems, methods, and instrumentalities associated with reconstructing a 3D representation of an anatomical structure based on a screen recording of a display while medical images of the anatomical structure are shown on the display. A plurality of medical images of the anatomical structure may be extracted from the screen recording, and one or more parameters for reconstructing the 3D representation of the anatomical structure based on the extracted medical images may be determined. The 3D representation of the anatomical structure may then be reconstructed based on the extracted medical images and the one or more determined parameters. From the 3D representation of the anatomical structure, abnormalities may be detected, and medical reporting may be performed using pre-trained machine-learning models.

### BACKGROUND

During clinical visits or through online portals provided by a medical facility, patients are often presented with medical images such as two-dimensional (2D) or three-dimensional (3D) medical scans of the patients' anatomies via a display (e.g., a computer screen). While the patients may view the medical images on the display, they may not have means to access those images directly, much less the ability to analyze the images and obtain alerts, indications, or diagnoses about an abnormality in those anatomies if the images were accessible to the patients.

### SUMMARY

Disclosed herein are systems, methods, and instrumentalities associated with reconstructing a 3D representation of an anatomical structure (e.g., a human organ) based on multiple screenshots that may be obtained via a screen recording. According to embodiments of the present disclosure, an apparatus may be configured to obtain multiple screenshots of a display (e.g., comprised in a video recording of the display) that may be captured while one or more medical representations (e.g., one or more medical scan images) of the anatomical structure are shown on the display. The apparatus may be further configured to extract a plurality of medical images of the anatomical structure from the multiple screenshots and determine one or more parameters for reconstructing a three-dimensional (3D) representation of the anatomical structure based on the plurality of extracted medical images. The apparatus may then reconstruct the 3D representation of the anatomical structure using the extracted medical images and the one or more determined parameters.

In examples, the multiple screenshots may be captured off the display of a desktop computer, a laptop computer, a tablet computer, or a mobile phone. In examples, the apparatus may receive the multiple screenshots from the tablet computer or the mobile phone. In examples, the apparatus may be the tablet computer or the mobile phone itself.

In examples, the apparatus may be further configured to detect, based on one or more pre-trained machine learning (ML) models and the 3D representation of the anatomical structure, an abnormality associated with the anatomical structure and provide an indication of the abnormality on the 3D representation of the anatomical structure. In examples, the apparatus may be further configured to generate, based on one or more pre-trained ML models and the 3D representation of the anatomical structure, a diagnostic report associated with the anatomical structure.

In examples, the one or more determined parameters may include a distance between two of the extracted medical images and/or a voxel size of the 3D representation. In examples, the apparatus may predict the distance between the two extracted medical images using a pre-trained ML model and determine the voxel size of the 3D representation based on the distance. In examples, the apparatus being configured to reconstruct the 3D representation of the anatomical structure based on the extracted medical images may comprise the apparatus being configured to identify one or more duplicates in the extracted medical images and exclude the one or more duplicates from the reconstruction of the 3D representation. In examples, the apparatus being configured to reconstruct the 3D representation of the anatomical structure based on the extracted medical images may comprise the apparatus being configured to adjust a size, an orientation, or an aspect ratio of at least one of the extracted medical images.

### BRIEF DESCRIPTION OF THE DRAWINGS

A more detailed understanding of the examples disclosed herein may be obtained from the following descriptions, given by way of example in conjunction with the accompanying drawings.

FIG. 1 is a simplified block diagram illustrating an example of reconstructing a 3D representation of an anatomical structure based on multiple screenshots of a display.

FIG. 2 is a simplified block diagram illustrating an example of extracting medical images from multiple screen shots.

FIG. 3 is a simplified block diagram illustrating an example of determining one or more 3D reconstruction parameters based on medical images extracted from multiple screenshots.

FIG. 4 is a flow diagram illustrating example operations associated with reconstructing a 3D representation of an anatomical structure based on a set of screenshots.

FIG. 5 is a flow diagram illustrating example operations associated with training an artificial neural network to perform one or more of the tasks described in embodiments of the present disclosure.

FIG. 6 is a simplified block diagram illustrating an example apparatus that may be configured to perform one or more of the tasks described in embodiments of the present disclosure.

### DETAILED DESCRIPTION

The present disclosure is illustrated by way of example, and not by way of limitation, in the accompanying drawings. A detailed description of illustrative embodiments will be provided with reference to these drawings. Although the embodiments may be described with certain details, it should be noted that the details are not intended to limit the scope of the disclosure.

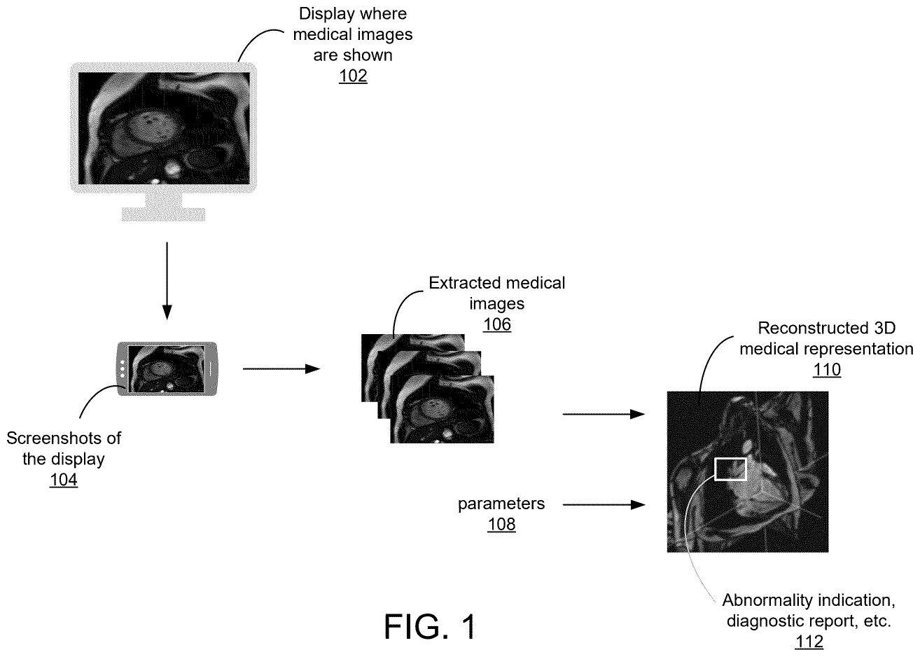

FIG. 1 illustrates an example of reconstructing a 3D representation of an anatomical structure (e.g., a human heart) based on multiple screenshots of a display. As shown in FIG. 1, the display (e.g., 102 of FIG. 1) may be a monitor or a screen such as the monitor of a desktop computer or a laptop computer, or the screen of a tablet computer or a mobile phone. The multiple screenshots 104 may be taken while a medical representation (e.g., a 2D or 3D computed tomography (CT) scan, magnetic resonance imaging (MRI) scan, or ultrasound scan) of the anatomical structure is shown on the display. For example, the multiple screenshots 104 may be part of a screen recording (e.g., a video) of a computer taken by a patient using a mobile device (e.g., a smart phone or tablet) during the patient's visit to a doctor's office, while medical scan images (e.g., CT, MRI, etc.) of the anatomical structure are shown to the patient using the computer. As another example, the multiple screenshots 104 may be recorded using a computer while a person is browsing medical scan images of the anatomical structure on the computer (e.g., via a patient portal accessible through a web browser installed on the computer). While the examples provided in this disclosure may treat the multiple screenshots 104 as part of a video recording, those skilled in the art will appreciate that the multiple screenshots 104 may also be taken individually, for example, as photos instead of the video recording.

According to embodiments of the present disclosure, the multiple screenshots 104 may be processed to extract a plurality of medical images 106 (e.g., which may also be referred to herein as slices) of the anatomical structure based on the multiple screenshots. The processing may be performed by a computing apparatus including, for example, a mobile device or a server. For instance, the processing may be performed by a mobile device (e.g., a tablet computer or a mobile phone) that was used to capture the multiple screenshots 104. The processing may also be performed by a server device (e.g., on a computing cloud) that may receive the multiple screenshots 104 from a mobile device used to capture the screenshots. The processing may also be performed by the computer on which the multiple screenshots 104 are recorded. As will be described in greater detail below, the processing may include identifying duplicated medical images from the multiple screenshots 104 and excluding those duplicate medical images from the plurality of medical images 106. The processing may also include adjusting the size, orientation, and/or aspect ratio of at least a subset of the plurality of medical images 106 (e.g., so that the plurality of medical images may be aligned for subsequent processing).

Since the plurality of medical images 106 is extracted from the multiple screenshots 104 of the display 102, the plurality of medical images 106 may correspond to those shown on the display 102 while the multiple screenshots 104 are taken. Once obtained, the plurality of medical images 106 may be used, together with one or more determined parameters 108, to reconstruct a 3D representation 110 of the anatomical structure. The one or more parameters 108 may include, for example, a slice thickness, a pixel spacing, physical dimensions covered by a slice, a voxel size of the 3D representation, a distance between two (e.g., any two) of the extracted medical images 106, and/or the like. As will be described in greater detail below, the one or more parameters 108 may be determined in different ways including, for example, extracting the parameters from the multiple screenshots 104 (e.g., via optical character recognition (OCR)) or predicting the parameters using one or more pre-trained machine-learning (ML) models.

The 3D representation of the anatomical structure 110 reconstructed based on the extracted medical images 106 and the one or more determined parameters 108 may correspond to the representation (e.g., 2D or 3D medical scan) shown on the display 102, and may be used for diagnostic purposes, treatment planning, and/or surgical navigation. For example, the 3D representation 110 may be used to detect an abnormality associated with the anatomical structure and provide an indication 112 (e.g., a bounding box, a segmentation mask, etc.) of the abnormality (e.g., on the 3D representation 110). As another example, the 3D representation 110 may be used to generate a diagnostic report associated with the anatomical structure based on features extracted from the 3D representation. As yet another example, the 3D representation 110 may be used to generate a treatment plan associated with the anatomical structure based on the extracted features of the 3D representation and/or a medical history of the concerned patient. As will be described in greater detail below, one or more of these tasks may be accomplished using pre-trained ML models.

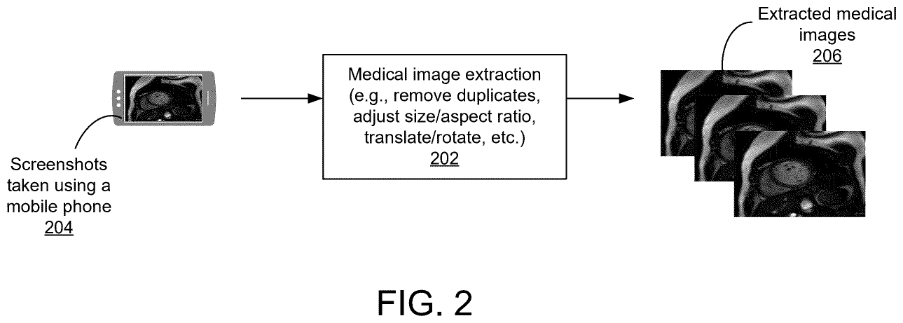

FIG. 2 illustrates an example of extracting medical images from a plurality of screen shots. As explained above, the screenshots (e.g., 204 of FIG. 2) may be captured using a mobile device while a medical representation (e.g., 2D or 3D medical scans) of an anatomical structure is shown on a display device. Due to the nature of such screen capturing, the screenshots 204 may not be directly suitable for reconstructing a 3D representation of the anatomical structure (e.g., the screenshots 204 may include duplicated images, images of different sizes or aspect ratios, images of poor quality, etc.). Therefore, the multiple screenshots 204 may be processed at 202 to extract a plurality of qualifying medical images 206 that may be used for the 3D reconstruction and, as part of the extraction process, one or more of the following may be performed.

The operations at 202 may include image preprocessing. For example, from a captured screenshot containing a medical image (e.g., a 2D medical image), four corners of the medical image may be determined (e.g., using a machine learning model trained for detecting visual features associated with the corners), and a bounding box may be derived based on the four corners and used to crop the medical image out from the screenshot. This way, only the medical image may be extracted from the screenshot while the remaining parts of the screenshot (e.g., such as unrelated GUI components showing on the screen) may be ignored. As another example, normalization may be applied to the medical images extracted from the screenshots 204 to ensure that they have consistent lighting and/or color correction to reduce discrepancies. As yet another example, resizing, upsampling, or downsampling may be performed to speed up processing while retaining sufficient detail. As yet another example, one or more filters (e.g., a Gaussian filter, a median filter, etc.) may be applied to reduce the noise in the images and to improve image quality.

The operations at 202 may include aligning the medical images extracted from the screenshots 204. The alignment may involve adjusting the geometric properties (e.g., size, aspect ratio, etc.) of the extracted medical images, and/or translating/rotating them to match corresponding points or features across multiple images to establish their relative positions and/or orientations. For example, distinct points of interest (e.g., keypoints) in each extracted image may be identified using feature detection algorithms such as SIFT (Scale-Invariant Feature Transform), SURF (Speeded-Up Robust Features), and/or ORB (Oriented FAST and Rotated BRIEF). For each detected keypoint, a descriptor may be computed to represent the feature (e.g., local appearance) around the keypoint (e.g., the descriptor may include a vector that represents the unique characteristics of the feature). The descriptors between images may then be matched to find corresponding points, and the closest matches may be determined based on one or more distance metrics (e.g., Euclidean distance).

In some examples, the matched keypoints may not all be correct due to noise, occlusion, repetitive textures, etc., so geometric verification may be performed to improve the alignment of the images. For example, an RANSAC (Random Sample Consensus) based filter matching technique may be used to estimate a robust transformation (e.g., a transformation matrix) that may align the images while discarding outliers. In some examples, if the extracted medical images are not already aligned on the same plane, rectification may be performed to bring corresponding points into alignment. This may be done, for example, where two screenshots are captured from slightly different viewpoints.

The operations at 202 may include detecting and excluding duplicated medical images (e.g., including medical images that are not exactly the same but are substantially similar) extracted from the screenshots 204. This may be accomplished by comparing the extracted medical images based on their visual content (e.g., rather than their file names or metadata). For example, the duplicated medical images may be identified using a hashing method with which a hash representing the visual content of an extracted medical image may be calculated and the duplicated medical images may be detected as having similar hashes. As another example, the duplicated medical images may be identified using a feature-based method with which key points and/or descriptors in an image may be identified and compared to find similar or duplicate images. As yet another example, the duplicated medical images may be identified using a deep learning-based approach that may utilize an artificial neural network such as, for example, a convolutional neural network (CNN). The neural network may be pre-trained to extract deep features from the medical images, and the features may then be compared using one or more similarity measures (e.g., cosine similarity, Euclidean distance, etc.) to detect duplicates in the medical images.

As shown in FIG. 1, certain parameter(s) 108 may be needed to reconstruct the 3D representation of the anatomical structure (e.g., based on the set of medical images 206 extracted from the multiple screenshots 204 shown in FIG. 2). The parameter(s) may include, for example, the number of medical images included in the extracted image set, a distance between consecutive images or slices in the extracted image set, the physical dimensions represented by a voxel in the 3D reconstruction, etc. In some examples, the parameter(s) may be obtained based on an external input (e.g., a user may provide the parameter(s)), while in other examples, the parameter(s) may be determined based on the set of medical images 206 or the screenshots 204 (e.g., by extracting information from the images or screenshots via OCR, or by predicting the parameter(s) using a pre-trained machine learning model).

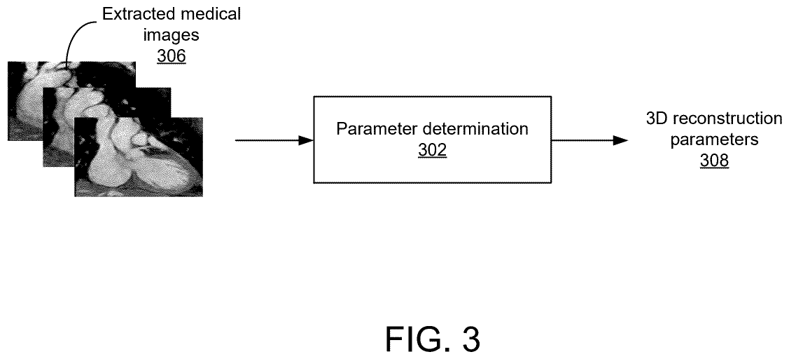

FIG. 3 illustrates an example process for determining one or more 3D reconstruction parameters (e.g., 308 of FIG. 3) based on medical images (e.g., 306 of FIG. 3) extracted from a screen recording (e.g., comprising multiple screenshots). The parameter determination may be performed at 302 and may use various techniques including conventional or deep learning based techniques. Using the determination of a voxel size for the 3D reconstruction as an example, such a parameter may be estimated based on the images 306 (e.g., 2D images) such as the physical dimensions of the images, the image resolution (e.g., the number of pixels in the 2D image by width x height), and/or a slice thickness that may be represented by the distance between consecutive 2D images or slices. The estimation may involve, for example, determining the pixel size in physical units and further determining the voxel size in the in-plane dimensions (x, y) and the z-dimension (e.g., the z-dimension measure may be determined based on the slice thickness mentioned above).

In examples, the voxel size may be predicted using a pre-trained ML model such as a deep learning (DL) model. Such an ML model may be implemented using various neural network architectures including, for example, a CNN, a ResNet, etc. The ML model may be trained in through a training process that may involve providing a plurality of 2D medical images to the ML model, forcing the ML model to make a prediction about a voxel size, comparing the predicted voxel size to corresponding ground truth, and adjusting the parameters of the ML model based on a loss between the predicted voxel size and the ground truth.

While the parameter determination at 302 is described using the voxel size as an example, those skilled in the art will appreciate that similar techniques may be applied to derive other parameters (e.g., the distance between consecutive images or slices) that may be needed for the 3D reconstruction.

Using the one or more parameters estimated according to FIG. 3 and a series of medical images (e.g., CT or MRI images) extracted from a screen recording (e.g., as shown in FIGS. 2), 3D medical image reconstruction may be performed to create a 3D representation of an anatomical structure (e.g., a patient's anatomy), as shown by 110 of FIG. 1. One or more of the following operations may be performed as part of the 3D reconstruction process. Other operations described above (e.g., image resizing/normalization, image registration or alignment, image denoising, etc.) may also be performed as part of the 3D reconstruction process. For example, the 3D reconstruction process may include identifying a region of interest (ROI) in the series of input images. This may be accomplished using various segmentation techniques such as, e.g., thresholding, region growing, or deep learning methods, with which the boundaries of organs, tissues, or other structures within each input image may be delineated. A 3D grid (e.g., voxel grid) may then be created to represent the volume that encompasses the input images or slices. The grid may be defined, for example, based on the dimensions and voxel size estimated from the 2D images (e.g., as discussed earlier). The intensity values of voxels between consecutive 2D slices may then be estimated, for example, using one or more interpolation techniques such as linear or cubic splines. As a result of the interpolation, a continuous 3D volume may be created, which may be visualized using various volume rendering techniques like ray-casting, maximum intensity projection (MIP), or surface rendering.

In examples, the 3D reconstruction may also include one or more post-processing steps to enhance or improve the reconstructed 3D representation. For example, one or more smoothing filters may be applied to reduce any artifacts from the reconstruction and enhance important features such as the edges of the anatomical structure. In addition, anatomical knowledge or machine learning-based correction methods may be used to adjust for errors in the segmentation or interpolation.

The 3D image (e.g., 110 of FIG. 1) of the anatomical structure reconstructed using the techniques described herein may be analyzed for diagnostic purposes, treatment planning, and/or surgical navigation. The analysis may be performed using conventional methods such as by performing volumetric measurements, distance calculations, morphological studies, or edge detection on the reconstructed 3D data. The analysis may also be performed using machine learning (e.g., deep learning) based techniques such as by training machine learning models to detect abnormalities in the anatomical structure and generate indications, reports, or treatment plans associated with the detected abnormalities (e.g., as shown by 112 of FIG. 1).

The machine learning (ML) model(s) used for the abnormality detection and/or report generation may include a CNN-based ML model, a transformer-based ML model, a large language model (LLM), and/or other types of deep learning models. The ML model(s) may be trained on actual patient medical images (e.g., 2D or 3D medical scans) to learn and extract features from those medical images, and recognize the features that may be associated with an abnormality. As will be described in greater detail below, the ML model(s) may be trained based on large volumes of clinical data and, more specifically, by splitting the data into training, validation, and test sets, training the model on the training set and validating it on the validation set (e.g. using appropriate loss functions, like binary cross-entropy, dice loss, etc.).

In examples, the abnormality detection and/or report generation may be accomplished using the reconstructed 3D representation in conjunction with other types of information (e.g., the data used to train the ML model(s) and generate the diagnoses may be multi-modal). These other types of information may include, for example, textual information such as a description of the symptoms experienced by a patient, lab reports of the patient, medical histories of the patient, etc. The information may also include other medical images of the patient such as, for example, MRI or CT images of the patient. The information may further include audio information such as the recording of a conversation between the patient and a physician, the patient's own narrated description of their health conditions, etc.

The ML models(s) may be trained using the multi-modal patient data and once trained, deployed to generate an output (e.g., 112 of FIG. 1) based on the multimodal patient data including the reconstructed 3D representation. One or more of the ML models may be implemented using an artificial neural network that may include multiple encoders and a decoder. Each of the encoders may be configured to receive a respective type of patient data and generate an encoded representation of the type of patient data (e.g., in the form of one or more vectors). The decoder may be configured to receive the encoded representations of the multimodal patient data (e.g., a concatenation of the encoded representations) and predict the output based on the encoded representations and/or an inquiry (e.g., a question posted by a patient). As described herein, the predicted output may include a medical decision such as a medical procedure (e.g., an MRI or CT scan) recommend for the patient, an indication of whether tumorous areas have been detected in the 3D representation, etc. The predicted output may also include a medical summary (e.g., a textual summary) of the health conditions of the patient generated based on the encoded representations.

In examples, one or more of the encoders may include a CNN comprising one or more convolutional layers, one or more pooling layers, and/or one or more fully connected layers. Each of the convolutional layers may include multiple kernels or filters with respective weights that may be configured to extract features from an input (e.g., a textual input or an image-based input). The convolution operations may be followed by batch normalization and/or an activation function (e.g., such as a rectified linear unit (ReLu) activation function), and the features extracted by the convolutional layers may be downsampled via the one or more pooling layers and/or fully connected layers to obtain a representation of the extracted features, for example, in the form of a feature vector. In examples, the network may employ a recurrent architecture to store hidden states associated with the input and feed the hidden states back into the convolutional layers (e.g., via one or more recurrent connections) of the encoder. This way, the encoder may, during feature encoding, utilize not only the current set of data samples passing through the network, but also previous data samples represented by the hidden states to derive a more accurate representation of the input data.

In examples, one or more of the encoders may include a transformer neural network with a built-in attention (e.g., self-attention) mechanism (e.g., comprising one or more self-attention layers) configured to detect the relationship between different parts of an input data sequence and learn the context (and thus the meaning) of the input data. These tasks may be accomplished, for example, based on query, key and value vectors or matrices.

In examples, the ML models described herein may include a vision-language model that may be trained to learn a mapping between visual and textual embeddings (e.g., between visual and textual features) from a dataset comprising paired images and textual descriptions. The training data may be obtained from various sources including, for example, the Internet (e.g., websites that may include images and descriptions of the content of the images), publicly accessible databases (e.g., figures and captions from repositories of academic publications), hospital records (e.g., radiology reports), etc. The training data may be pre-processed, for example, to ensure that it is in a suitable format for the training. The pre-processing may, for example, include resizing the images, tokenizing the text, creating pairs of image-text inputs, etc. The pre-processing may also include augmenting the training data (e.g., by varying the textual descriptions to increase the diversity of the training dataset) to improve the robustness and accuracy of the vision-language model.

The vision-language model may include a vision encoding portion (e.g., implemented via a vision encoder) and a text encoding portion (e.g., implemented via a text encoder). In examples, the vision encoder may utilize a vision transformer architecture designed to extract image features from input images, while the text encoder may be implemented using a regular transformer architecture designed to extract text features from textual descriptions. The image features and text features may then be aligned (e.g., mapped to each other) in a joint embedding space (e.g., through concatenation or some other suitable fusion techniques) to capture the relationships between the visual and textual information. In examples, the vision encoder and the text encoder may be trained first (e.g., separately) on a large number of images and textual descriptions, respectively, and then fine-tuned using an application specific dataset (e.g., a certain type of medical scan images) and/or based on a specific downstream task (e.g., medical image classification).

The training may allow the vision-language model to acquire an understanding of the relationships between certain visual and textual embeddings or features such that, when given an image (e.g., the 3D representation 110 of FIG. 1) as an input, the vision-language model may extract visual features from the input and generate a coherent and informative explanation (e.g., a diagnostic report) of the visual information contained in the input by relating the extracted visual features to corresponding textual features in the learned joint embedding space.

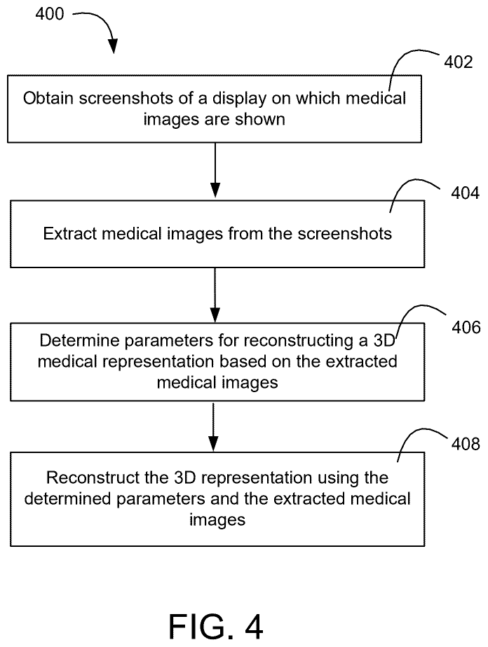

FIG. 4 illustrates an example procedure 400 that may include one or more of the operations described herein. The procedure 400 may be performed by various devices, independently or collaboratively. For example, the procedure 400 may be performed by a server (e.g., in a computing cloud) configured to receive screen recordings of a display from another device (e.g., a mobile device). The procedure 400 may also be performed by the device (e.g., a mobile device or a desktop computer) used to capture the screenshots.

As shown in FIG. 4, the procedure 400 may include obtaining, at 402, multiple screenshots of a display, wherein the multiple screenshots may be captured while a medical representation of an anatomical structure is shown on the display. The procedure 400 may further include extracting a plurality of medical images (e.g., 2D medical images) of the anatomical structure from the multiple screenshots at 404, and determining, at 406, one or more parameters for reconstructing a 3D representation of the anatomical structure using the plurality of extracted medical images. The procedure 400 may additionally include reconstructing the 3D representation of the anatomical structure at 408 based on the plurality of medical images extracted from the screenshots and the one or more determined parameters.

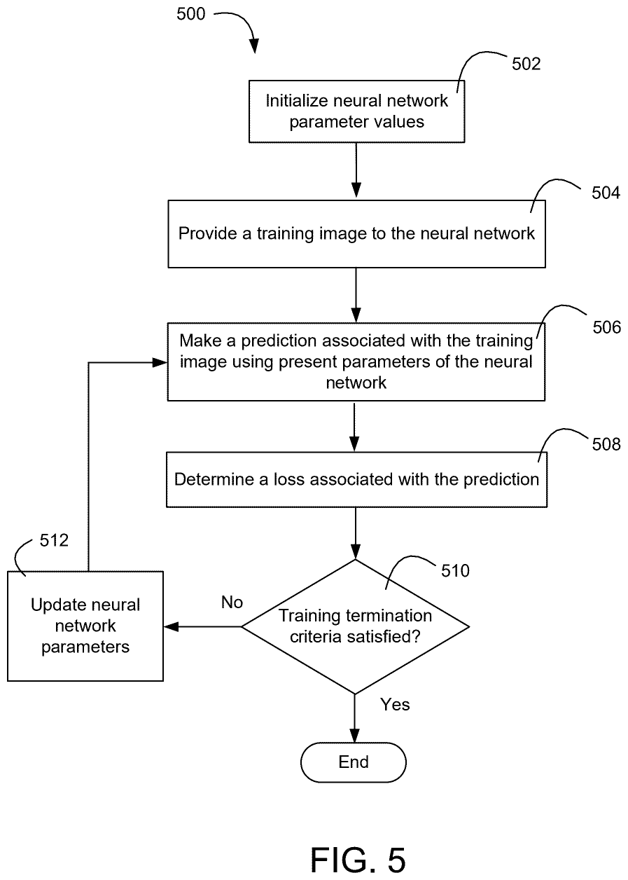

FIG. 5 illustrates example operations 500 that may be associated with training an artificial neural network (e.g., which may be configured to implement one or more of the ML models described herein) to perform one or more of the tasks described herein. As shown in FIG. 5, the training operations 500 may include initializing the operating parameters of the neural network (e.g., weights associated with various layers of the neural network) at 502, for example, by sampling from a probability distribution or by copying the parameters of another neural network having a similar structure. The training operations may further include providing an input (e.g., a reconstructed 3D medical image) to the neural network at 504 and causing the neural network to make a prediction (e.g., about a classification label, a segmentation mask, etc.) using presently assigned network parameters at 506. At 508, the training operations may include determining a loss associated with the prediction, for example, based on a difference between the prediction and corresponding ground truth. At 510, the training operations may further include determining whether one or more training termination criteria have been satisfied. For example, the training termination criteria may be determined to have been satisfied if the difference between the prediction and the ground truth falls below a predetermined threshold value. If the determination at 510 is that the training termination criteria are satisfied, the training may end. Otherwise, the presently assigned network parameters may be adjusted at 512, for example, by backpropagating a gradient descent of the loss through the network, before the training returns to 506.

For simplicity of explanation, the training operations are depicted and described herein with a specific order. It should be appreciated, however, that the training operations may occur in various orders, concurrently, and/or with other operations not presented or described herein. Furthermore, it should be noted that not all operations that may be included in the training process are depicted and described herein, and not all illustrated operations are required to be performed.

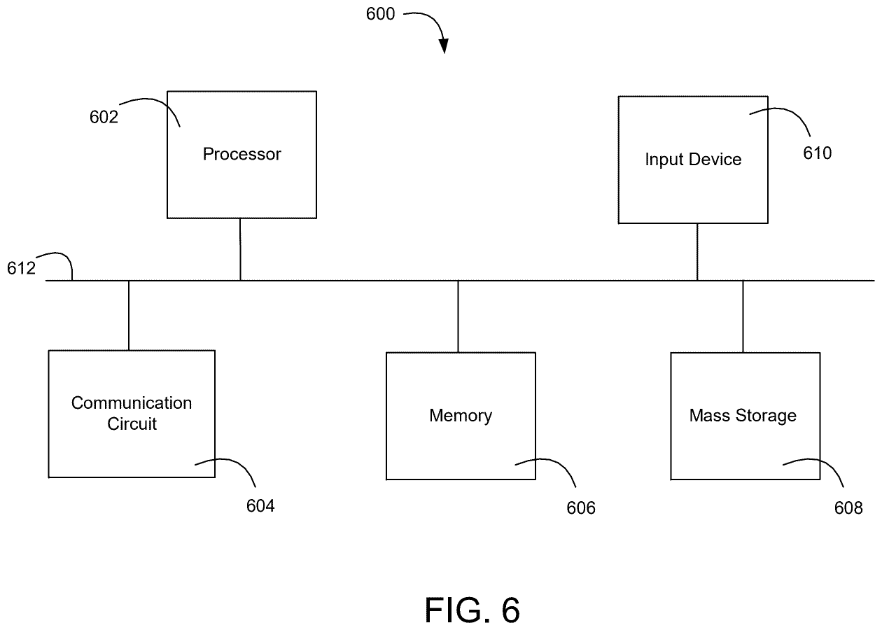

The systems, methods, and/or instrumentalities described herein may be implemented using one or more processors, one or more storage devices, and/or other suitable accessory devices such as display devices, communication devices, input/output devices, etc. FIG. 6 is a block diagram illustrating an example apparatus 600 that may be configured to perform the tasks described herein. As shown, apparatus 600 may include a processor (e.g., one or more processors) 602, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

The communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). The memory 606 may include a storage medium (e.g., a non-transitory storage medium) configured to store machine-readable instructions that, when executed, cause the processor 602 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. The mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of the processor 602. The input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked, or clustered) with other computation devices to perform the functions described herein. And even though only one instance of each component is shown in FIG. 6, a skilled person in the art will understand that apparatus 600 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

The term “computer-readable storage medium” used herein may include any tangible medium that is capable of storing or encoding a set of instructions for execution by a computer that cause the computer to perform any one or more of the methods described herein. The term “computer-readable storage medium” used herein may include, but not be limited to, solid-state memories, optical media, and magnetic media.

1. An apparatus, comprising:
one or more processors configured to:
obtain multiple screenshots of a display, wherein the multiple screenshots were captured by a mobile phone or a tablet while a medical representation of an anatomical structure is shown on the display;
extract a plurality of medical images of the anatomical structure from the multiple screenshots, wherein, as part of the extraction, the one or more processors are configured to:
crop, from the multiple screenshots, respective areas that correspond to the medical representation of the anatomical structure, wherein the cropping is performed based at least on visual features extracted from the multiple screenshots; and
process the cropped areas to derive the plurality of medical images, wherein, during the processing, a size, an orientation, or an aspect ratio of at least one of the cropped areas is adjusted and at least one duplicate in the cropped areas is detected and eliminated;
determine one or more parameters for reconstructing a three-dimensional (3D) representation of the anatomical structure based on the plurality of extracted medical images, wherein the one or more parameters include a distance between two of the plurality of extracted medical images and a voxel size for the 3D representation, the distance predicted using a pre-trained machine learning (ML) model, the voxel size determined based on the distance; and
reconstruct the 3D representation of the anatomical structure based on the plurality of extracted medical images and the one or more determined parameters.

2. The apparatus of claim 1, wherein the multiple screenshots were captured off the display of a desktop computer, a laptop computer, the tablet, or the mobile phone.

3. The apparatus of claim 2, wherein the multiple screenshots were captured off the display of the tablet or the mobile phone, and wherein the one or more processors are configured to receive the multiple screenshots from the tablet or the mobile phone.

4. The apparatus of claim 3, wherein the apparatus is the tablet or the mobile phone.

5. The apparatus of claim 1, wherein the multiple screenshots were captured as a screen recording of the display.

6. The apparatus of claim 1, wherein the one or more processors are further configured to detect, based on one or more pre-trained machine learning (ML) models and the 3D representation of the anatomical structure, an abnormality associated with the anatomical structure and provide an indication of the abnormality on the 3D representation of the anatomical structure.

7. The apparatus of claim 1, wherein the one or more processors are further configured to generate, based on one or more pre-trained machine learning (ML) models and the 3D representation of the anatomical structure, a diagnostic report associated with the anatomical structure.

8-11. (canceled)

12. A method for reconstructing a three-dimensional (3D) representation of an anatomical structure, the method comprising:
obtaining multiple screenshots of a display, wherein the multiple screenshots were captured by a mobile phone or a tablet while a medical representation of the anatomical structure is shown on the display;
extracting a plurality of medical images of the anatomical structure from the multiple screenshots, wherein the extraction comprises:
cropping, from the multiple screenshots, respective areas that correspond to the medical representation of the anatomical structure, wherein the cropping is performed based at least on visual features extracted from the multiple screenshots; and
processing the cropped areas to derive the plurality of medical images, wherein, during the processing, a size, an orientation, or an aspect ratio of at least one of the cropped areas is adjusted and at least one duplicate in the cropped areas is detected and eliminated;
determining one or more parameters for reconstructing the 3D representation of the anatomical structure based on the plurality of extracted medical images, wherein the one or more parameters include a distance between two of the plurality of extracted medical images and a voxel size for the 3D representation, the distance predicted using a pre-trained machine learning (ML) model, the voxel size determined based on the distance; and
reconstructing the 3D representation of the anatomical structure based on the plurality of extracted medical images and the one or more determined parameters.

13. The method of claim 12, wherein the multiple screenshots were captured off the display of a desktop computer, a laptop computer, the tablet, or the mobile phone.

14. The method of claim 12, wherein the multiple screenshots were captured as a screen recording of the display.

15. The method of claim 12, further comprising detecting, based on one or more pre-trained machine learning (ML) models and the 3D representation of the anatomical structure, an abnormality associated with the anatomical structure and providing an indication of the abnormality on the 3D representation of the anatomical structure.

16. The method of claim 12, further comprising generating, based on one or more pre-trained machine learning (ML) models and the 3D representation of the anatomical structure, a diagnostic report associated with the anatomical structure.

17-19. (canceled)

20. A non-transitory storage medium comprising instructions that, when executed by a processor included in a computing device, cause the processor to implement the method of claim 12.
